(19) United States
(12) Patent Application Publication
(10) Pub. No.: US 2007/0003596 A1
(43) Pub. Date: Jan. 4, 2007

(54) DRUG DEPOT FOR PARENTERAL, IN PARTICULAR INTRAVASCULAR, DRUG RELEASE

(76) Inventors: Michael Tittelbach, Nurnberg (DE); Torben Bertsch, Nurnberg (DE); Ralf Bock, Erlangen (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 11/426,208

(22) Filed: Jun. 23, 2006

(30) Foreign Application Priority Data
Jul. 4, 2005 (DE) 10 2005 031 868.1

(51) Int. Cl. A61K 31/277; A61K 33/06; A61F 2/02

(52) U.S. Cl. 424/426; 514/521; 424/682

(57) ABSTRACT

A drug depot suitable for the parenteral, in particular intravascular, release of at least one drug which may be present as a base or corresponding protonised salt, wherein the base has a pKb value ranging from 2 to 6. The drug depot contains elementary magnesium in a biocorrodible form for this purpose.

### FIELD OF THE INVENTION

The invention relates to a drug depot for parenteral, in particular intravascular, drug release.

### BACKGROUND OF THE INVENTION

A major concern in pharmaceutical technology is the development of drugs of pharmaceutical forms for controlled drug release, particularly for the delay in release, to provide sustained, and also uniform, dosage of a drug over a longer period. This is achieved, for example, by the supply of preliminary stages of the drug that are initially ineffective and can only be activated in the body, or by the addition of excipients that delay the resorption of the drug. Furthermore, parenteral application of the drug is also conceivable, for example as an injected microcrystalline suspension or by implanting a biocorrodible drug depot which gradually releases the drug to be injected.

Drug depots are blanks of biocorrodible materials that are millimetres or centimetres in size, for example biocorrodible polymers of natural origin, e.g., poly-DL-lactide-co-glycolides, or polymers that are obtained by synthetic methods. The drug is mixed with the biocorrodible material, applied as a coating or incorporated in an envelope of the material. After implantation the drug is gradually released as the material degrades.

Release behaviour of the drug depends very much on the interaction between the drug and the biocorrodible material used, as well as on its products of degradation, if applicable. In other words, this type of interaction will have to be considered if parenteral, in particular intravascular drug release is to be optimised. Consideration must also be given to the point of release in the body, since considerable local differences may be displayed in the factors influencing the release, such as hydrophily/hydrophobia, pH value, flow ratios or oxygen content.

### SUMMARY OF THE INVENTION

The feature of this invention is to supply a drug depot suitable for the parenteral, in particular intravascular, release of at least one drug—which may be present as a base or a protonised salt corresponding to it, the base having a pKb value ranging from 2 to 6.

The drug depot according to the invention, for parenteral, in particular intravascular drug release, achieves this feature. The drug depot contains elementary magnesium in a biocorrodible form. The invention is based on the knowledge that drugs which may be present as a base or protonised salt corresponding to it, the base having a pKb value ranging from 2 to 6, are present in the salt form, at least predominantly, in a physiological environment. If the drug is present as a base, its hydrophily increases considerably with the conversion of the drug to its salt form, so that the release of the drug in a physiological environment is accelerated. If the drug is already present in the drug depot in its salt form, e.g., because salts can generally be more readily processed, and because the base form has a liquid or oily consistency at room temperature, the drug is also released very quickly in a physiological environment.

In a physiological environment, particularly in blood, the elementary magnesium present according to the invention is now degraded to highly basic magnesium hydroxide. This ensures that the salt form of the drug is again deprotonised and the equilibrium is displaced in the direction of the more hydrophobic base form of the drug. The resorption behaviour of the drug in the body can be influenced with the transition from the hydrophilic salt form of the drug to the hydrophobic base form of the drug. If the drug depot is adapted for intravascular drug release, fast release of the hydrophilic salt form of the drug into the blood can therefore be prevented or at least reduced. Moreover, if the drug depot remains on the vessel wall, it is assumed that specific application in the direction of the vessel wall is possible, for this is much more hydrophobic in nature than the blood flowing in the lumen of the vessel. This should assist penetration of the more hydrophobic base form of the drug.

The pKb value is defined as the negative decadic logarithm of the dissociation equilibrium B+H₂O⇄BH⁺+OH⁻ of the electrolytic dissociation, B standing for base. The basicity of the drug at room temperature is used to express this.

The term drug refers here to a substance which can be used as a therapeutic medicine for influencing conditions or functions of the body. The drug performs a basic function is preferably an organic nitrogen compound, but could also be a sulphur or phosphorus compound. The drug is further characterised in that it is present particularly in the blood, predominantly in the form of the corresponding salt, due to protonisation. The normal pH value in the arterial blood is approximately 7.40, and in venous blood approximately 7.37. Drugs with a pKb value ranging from 2 to 6, preferably ranging from 3 to 6, are suitable for the purposes according to the invention.

Drugs of the aforementioned type preferably contain aliphatic amines. It is particularly preferable for the drug to be Verapamil (5-[N-(3,4-Dimethoxyphenethyl)-N-methylamino]-2-(3,4-dimethoxyphenyl)-2-isopropyl-valeronitrile). Verapamil is a calcium antagonist and has a pKb value of 5.4. Verapamil is used preferably as hydrochloride for manufacturing the drug depot because the salt can be processed more effectively than substrate of the non-ionic amine, which is oily at room temperature.

A biocorrodible form of elementary magnesium is present when degradation of the magnesium takes place after implantation of the drug depot due to internal physical processes. The degradation is conditioned mainly by hydrolytic processes during which the strong base magnesium hydroxide is formed. The biocorrosion of the elementary magnesium is preferably more than 90% by weight complete, related to the proportion to the proportion of the total elementary magnesium present before the implantation, after a maximum of 6 months, in particular preference 3 months.

It is also preferable for the elementary magnesium to form part of a biocorrodible magnesium alloy, by which is meant, in this case, an alloy in which magnesium has a proportion by weight of at least 50%. The biocorrodible magnesium alloy is preferably an alloy with the composition yttrium 3.7-5.5% by weight, rare earths 1.5-4.4% by weight, and the residue <1% by weight, magnesium representing the proportion of the alloy making up 100% by weight. The magnesium alloys are characterised by their easy processability and their favourable degradation behaviour for the purposes of the invention.

By varying a molar ratio of the magnesium hydroxide formed with the degradation of magnesium for the drug, the release behaviour can be influenced. The proportion of the magnesium hydroxide is increased compared with the drug, the release in a hydrophilic medium is delayed. It is clear that a molar ratio of the magnesium hydroxide for the drug, formed with the degradation of magnesium should, in particular preference for the purpose of the invention, should preferably in the range of 1:1 to 50:1, in particular preference in the range of 1:1 to 10:1, (i) over the period in which the equilibrium between the hydrophilic salt form of the drug can be displaced to the hydrophobic base form of the drug, and (ii) limited locally to the point of retardation of the drug. This can ensure that the retardation of the drug takes place in delayed fashion according to the invention after implantation of the drug depot.

Preferably the above-mentioned period extends over 1 to 90 days, in particular 3 to 30 days, commencing with the implantation of the drug depot in a blood vessel. The point of retardation refers to the area of the drug depot at which the body medium (generally blood) comes into contact with the drug.

The point of retardation will generally correspond to an interface which is produced between the body medium and the solid or oily drug or a formulation containing the drug. A concentration of the magnesium hydroxide and hence a molar ratio of the same for the drug at the point of retardation depends essentially on (1) a rate at which the elementary magnesium is converted to magnesium hydroxide, and (ii) a local proximity between a place of degradation of the magnesium and the point of retardation. Here again the rate of conversion depends mainly on the form in which the elementary magnesium is present. The conversion of a biocorridible magnesium alloy is therefore delayed compared with pure magnesium. Moreover a composition of suitable magnesium alloys determines the rate of conversion of the magnesium contained in them, i.e., by adjusting the alloy composition, optimisation can be achieved so that the desired release behaviour of the drug is achieved. The further apart the point of degradation of the magnesium is from the point of retardation, the higher must be the conversion of the magnesium to magnesium hydroxide in order to set the desired concentration ratios at the point of retardation.

A aspect of the invention relates to a method for manufacturing a drug depot for the parenteral, in particular intravascular drug release, which comprises the following step: Mixing or coating elementary magnesium in a biocorrodible form with at least one drug, which may be present as a base or as a corresponding protonised salt, the base having a pKb value ranging from 2 to 6, and with further excipients if necessary.

The elementary magnesium—whether in pure form or as biocorrodible magnesium alloy—may therefore be mixed as power or grain with the drug, and with further excipients if necessary. Using known forming techniques, the mixture is used to manufacture a blank which has a form and condition suitable for the intended purpose.

Alternatively the mixture may be applied as a coating on a substrate, for example an endovascular drug depot, pacemaker or the electrodes of an electrotherapeutic implantate. The coating according to this exemplary embodiment then acts as a drug depot within the meaning according to the invention. Excipients may include all current additives of known pharmaceutical formulations which are used to assist in the manufacture of the blank or coating.

According to a further exemplary embodiment the drug depot may incorporate a solid body of magnesium or a biocorrodible magnesium alloy. According to this variant the drug—if necessary with further excipients—is applied as a coating to the body. This may be carried out, for example, by spraying or immersing the body in or with a solution of the drug in a suitable solvent.

A third aspect of the invention relates to the use of elementary magnesium—whether in pure form or as a biocorrodible magnesium alloy—for manufacturing a drug depot for the parenteral, in particular intravascular release, of at least one drug, which may be present as a base or corresponding protonised salt, the base having a pKb value ranging from 2 to 6. According to the level of knowledge of the applicant, elementary magnesium, preferably in the form of a biodegradable magnesium alloy, has not previously been used for drug depots for parenteral application of basic drugs.

### BRIEF DESCRIPTION OF THE DRAWINGS

FIG. 1 shows, in a highly schematised form, an exemplary embodiment of a drug depot according to the present invention for vascular application;

FIG. 2 shows a section through part of a drug depot according to a first variant; and

FIG. 3 shows a section through part of a drug depot according to a second variant.

### DETAILED DESCRIPTION OF THE INVENTION

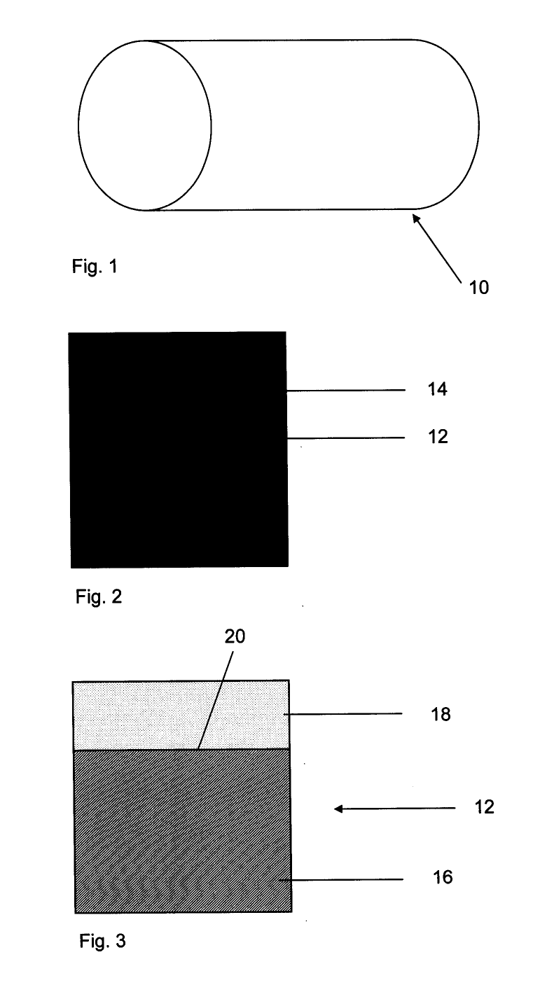

FIG. 1 shows an exemplary embodiment of a drug depot 10 for the parenteral, intravascular drug release, which is formed from a magnesium alloy with the composition yttrium 3.7 to 5.5% by weight, rare earths 1.5 to 4.4% by weight and residue <1% by weight, magnesium representing the proportion of the alloy making up the 100% by weight. Drug depot 10 is optimised for use in a blood vessel, i.e., it has a tubular basic body through the inside of which blood is able to flow. Drug depot 10 may be anchored by suitable means, e.g., small hooks or spikes, in a vessel wall of the blood vessel.

Drug depot 10 is wetted with an approximately 1 molar solution of the hydrochloride of Verapamil in acetone, and the solvent is then evaporated under reduced pressure. The process is repeated until a molar ratio of the magnesium to the Verapamil hydrochloride is approximately in the range of 30:1 to 50:1. Even after the coating one surface of the solid basic body of the magnesium alloy is still accessible, enabling degradation of the magnesium alloy to take place with the release of magnesium hydroxide. For the ratio of magnesium to the drug indicated, it is assumed that a molar ratio of the magnesium hydroxide formed with the degradation of magnesium to the drug can be desired to the desired value. A period in which the equilibrium between the hydrophilic salt form of Verapamil can be displaced to the hydrophobic base form of Verapamil should be approximately 3 to 30 days, commencing with the implantation of the drug depot in a blood vessel. The molar ratio of magnesium hydroxide to the Verapamil should in this case be 1:1 to 10:1 at the point of retardation over the period mentioned.

FIGS. 2 and 3 each show a section through part 12 of a drug depot according to two variants. A geometry of part 12 shown is only of subordinate importance and must be adapted according to the structural requirements of the drug depot. Only the basic structure will be demonstrated here.

In the variant according to FIG. 2, part 12 of the drug depot shown consists of a largely homogeneous mixture of a drug (denoted by the grains) and elementary magnesium as a matrix surrounding this drug (denoted by the clearances between the grains). For example, the drug is Verapamil in the form of its hydrochloride. After the implantation a surface 14 is in contact with the body medium, generally blood, so that a local concentration of magnesium hydroxide is increased at an interface between surface 14 of the dug depot and the body medium. This in turn causes the Verapamil to be transferred from the saline hydrochloride to its oily non-ionic form. The latter can only be dissolved to a negligible extent in an aqueous medium such as blood.

In the variant according to FIG. 3 part 12 of the drug depot shown is designed so that it has two elements. A solid basic body 16 of a biocorrodible magnesium alloy is covered with a porous coating 18 which contains the drug.

The drug may, for example again be Verapamil which is present as hydrochloride. The drug may again be Verapamil, for example, which is present as hydrochloride. Normal substrates for drugs are also added to coating 18, in which case sufficient porosity of coating 18 must be guaranteed.

After the implantation the body medium can only penetrate the basic body 16 via coating 18, i.e., the lateral surfaces shown in FIG. 3 and the bottom of the solid basic body 16 are correspondingly structurally inaccessible (e.g., the drug depot is of spherical design with an inner core as the basic body). A surface 20 of basic body 16 is in contact with the body medium after the implantation.

Consequently there is a conversion of the magnesium to magnesium hydroxide on surface 20. This will be distributed by diffusion in coating 18 so that a local concentration of magnesium hydroxide is increased at the interface between a surface of coating 18 containing the hydrochloride of Verapamil. This in turn causes the Verapamil to be transferred from the saline hydrochloride to the oily non-ionic form. The latter can only be dissolved to a negligible extent in an aqueous medium such as blood.

1. A drug depot for parenteral, in particular intravascular, drug release, comprising:
(a) at least one drug present as a base or as a corresponding protonised salt, the base having a pKb value ranging from 2 to 6; and,
(b) elementary magnesium in a biocorrodible form.

2. The drug depot of claim 1, wherein the elementary magnesium is a constituent of a magnesium alloy.

3. The drug depot of claim 1, wherein the drug is verapamil.

4. The use of elementary magnesium in a biocorrodible form for manufacturing a drug depot for parenteral, in particular intravascular, release of at least one drug, the at least one drug being present as a base or as a corresponding protonised salt, the base having a pKb value ranging from 2 to 6.

5. A method for the manufacture of a drug depot for the parenteral, in particular intravascular drug releases, comprising:
mixing or coating of elementary magnesium in a biocorrodible form with at least one drug present as a base or corresponding protonised salt;
wherein the base has a pKb value ranging from 2 to 6, and with further excipients if necessary.

6. The drug depot of claim 1, wherein the drug is verapamil.
